(19) United States
(12) Patent Application Publication
(10) Pub. No.: US 2017/0251962 A1
(43) Pub. Date: Sep. 7, 2017

(54) WEARABLE DEVICE, METHOD FOR GENERATING NOTIFICATION INFORMATION, AND SYSTEM

(71) Applicant: SEIKO EPSON CORPORATION, Tokyo (JP)

(72) Inventor: Kazuhiro SHIHO, Shiojiri-shi (JP)

(73) Assignee: SEIKO EPSON CORPORATION, Tokyo (JP)

(21) Appl. No.: 15/445,082

(22) Filed: Feb. 28, 2017

(30) Foreign Application Priority Data
Mar. 2, 2016 (JP) 2016-039685

(51) Int. Cl. A61B 5/1455; A61B 5/0205; A61B 5/00

(52) U.S. Cl. CPC A61B 5/14551; A61B 5/742; A61B 5/6803; A61B 5/0205; A61B 5/0004; A61B 2560/0242

(57) ABSTRACT

A wearable device includes an information acquisition section which acquires environmental information including at least one of location information of a user, atmospheric pressure information, and weather information, a sensor which acquires arterial oxygen saturation information, a processing section which generates notification information associated with an arterial oxygen saturation based on the environmental information and the arterial oxygen saturation information, and an output section which outputs the notification information.

### BACKGROUND

1. Technical Field

The present invention relates to a wearable device, a method for generating notification information, and a system.

2. Related Art

An oxygen concentration in air is low (an oxygen partial pressure is low) at a high altitude, and therefore, in an activity at a high altitude such as mountain climbing, symptoms of altitude sickness are sometimes developed due to a lack of experience or the climber's own overconfidence. Since apraxia, alpine accident, etc. due to symptoms of altitude sickness increase, a demand for a method for performing an activity at a high altitude safely is large.

For example, JP-A-2013-34767 (Patent Document 1) discloses a method for determining the physical condition of a user during mountain climbing based on the altitude.

The response to the altitude (height) or the change in the altitude differs greatly among individuals. For example, it is said that a general altitude which may cause altitude sickness is about 2400 m to 2500 m, however, some people sometimes develop altitude sickness when the altitude is about 2000 m. Further, even in the case of the same user, the risk of development of symptoms of altitude sickness changes depending on the physical condition at that time, the degree of high-altitude acclimatization, or the like. That is, it is difficult to accurately determine the risk of a load applied to the body of a user based only on the altitude.

Further, a pulse oximeter capable of measuring an arterial oxygen saturation (in particular, SpO₂ which is a percutaneous arterial oxygen saturation) has been widely known. SpO₂ serves as an index which indicates the amount of oxygen capable of being taken in the body of a user. Therefore, it is considered that by using the information of SpO₂, the risk of an activity at a high altitude can be more accurately determined than using altitude information.

However, many of the currently widely used pulse oximeters are used by being attached to a fingertip and therefore are not suitable for continuous wearing. Due to this, the device interferes with the activity of a user such as mountain climbing, or the user has already been in a hypoxic state for a long period of time when measurement is performed, and therefore symptoms of altitude sickness may not be able to be prevented. Further, even in the case of a healthy user, the value of SpO₂ changes depending on the environment. Therefore, in the case where only the information of SpO₂ is used, it is difficult to generate appropriate information and make a notification.

### SUMMARY

An advantage of some aspects of the invention is to provide a wearable device which generates and outputs notification information for giving an appropriate notification to a user using environmental information and arterial oxygen saturation information, a method for generating notification information, a system, and the like.

An aspect of the invention relates to a wearable device which includes an information acquisition section which acquires environmental information including at least one of location information of a user, atmospheric pressure information, and weather information, a sensor which acquires arterial oxygen saturation information, a processing section which generates notification information associated with an arterial oxygen saturation based on the environmental information and the arterial oxygen saturation information, and an output section which outputs the notification information.

The wearable device according to the aspect of the invention generates notification information based on environmental information and arterial oxygen saturation information. By using the wearable device, measurement can be performed frequently (in a narrow sense, continuous measurement) without inhibiting the activity of a user. In addition, by also using environmental information for generating notification information, it becomes possible to generate and output notification information according to the condition of the user.

In the wearable device according to the aspect of the invention, the output section may be a display section, and the display section may display at least one of advice information associated with the arterial oxygen saturation and alert information associated with the arterial oxygen saturation as the notification information.

According to this configuration, it becomes possible to display at least one of advice information and alert information on the display section of the wearable device.

In the wearable device according to the aspect of the invention, the display section may display the notification information, the environmental information, and the arterial oxygen saturation information.

According to this configuration, it becomes possible to display environmental information and arterial oxygen saturation information in addition to notification information.

In the wearable device according to the aspect of the invention, the advice information may be advice information associated with the continuation of the exercise or the load of the exercise of the user.

According to this configuration, it becomes possible to generate and output advice information associated with the exercise of the user as notification information.

In the wearable device according to the aspect of the invention, the processing section may determine which range of a first range to an N-th (N is an integer of 2 or more) range the arterial oxygen saturation falls in based on the arterial oxygen saturation information, and when the arterial oxygen saturation is determined to fall in an i-th (i is an integer that satisfies the following relationship: 1≦i≦N) range, the processing section may generate i-th advice information.

According to this configuration, it becomes possible to generate advice information among a plurality of pieces of advice information according to the range of the arterial oxygen saturation.

In the wearable device according to the aspect of the invention, the processing section may change the arterial oxygen saturation corresponding to at least one range of the first range to the N-th range according to the environmental information.

According to this configuration, it becomes possible to generate and output advice information according to environmental information.

In the wearable device according to the aspect of the invention, the output section may be a communication section, and the communication section may transmit alert information associated with the arterial oxygen saturation as the notification information.

According to this configuration, it becomes possible to transmit alert information to a device which is different from the wearable device.

In the wearable device according to the aspect of the invention, the device may further include a memory section which stores history information of the arterial oxygen saturation information, and the processing section may generate the notification information based on the history information.

According to this configuration, it becomes possible to generate notification information based on history information.

In the wearable device according to the aspect of the invention, the history information may be information in which each piece of arterial oxygen saturation information and the environmental information are associated with each other.

According to this configuration, it becomes possible to generate notification information based on history information in which arterial oxygen saturation information and environmental information are associated with each other.

In the wearable device according to the aspect of the invention, the history information may be information in which each piece of arterial oxygen saturation information, the environmental information, and biological information of the user are associated with one another.

According to this configuration, it becomes possible to generate notification information based on history information in which arterial oxygen saturation information, environmental information, and biological information are associated with one another.

Another aspect of the invention relates to a system which includes the wearable device according to the aspect of the invention, and a portable terminal device or an information processing device which makes a notification based on the notification information received from the wearable device.

Another aspect of the invention relates to a method for generating notification information using a wearable device, which includes acquiring environmental information including at least one of location information of a user, atmospheric pressure information, and weather information, generating notification information associated with an arterial oxygen saturation based on arterial oxygen saturation information from a sensor and the environmental information, and outputting the notification information.

Another aspect of the invention relates to a program which causes a computer to execute acquiring environmental information including at least one of location information of a user, atmospheric pressure information, and weather information, generating notification information associated with an arterial oxygen saturation based on arterial oxygen saturation information from a sensor and the environmental information, and outputting the notification information.

Another aspect of the invention relates to a wearable device, wherein environmental information including at least one of location information of a user, atmospheric pressure information, and weather information, information associated with an arterial oxygen saturation of the user, pulse wave information of the user, and at least one of advice information associated with the arterial oxygen saturation and alert information associated with the arterial oxygen saturation are displayed on a display section.

In the wearable device according to the aspect of the invention, the advice information may be information associated with the continuation of the exercise or the load of the exercise of the user.

In the wearable device according to the aspect of the invention, the alert information may be information associated with a pace of the user.

### BRIEF DESCRIPTION OF THE DRAWINGS

The invention will be described with reference to the accompanying drawings, wherein like numbers reference like elements.

FIG. 1 shows a system configuration example of a wearable device.

FIG. 2 shows an example of an external appearance of a wearable device.

FIG. 3 shows an example of an external appearance of a wearable device.

FIG. 4 shows an example of connection between a wearable device and another device.

FIG. 5 shows an example of connection between a wearable device and another device.

FIG. 6 is a view for explaining the principle of a method for acquiring arterial oxygen saturation information.

FIG. 7 is a relational view of the altitude and the value of SpO₂ of a healthy subject.

FIG. 8 shows an example of correspondence between the range of SpO₂ and advice information.

FIG. 9 shows another example of correspondence between the range of SpO₂ and advice information.

FIG. 10 shows another example of correspondence between the range of SpO₂ and advice information.

FIG. 11 is a flowchart illustrating notification information generation processing based on the altitude and SpO₂.

FIG. 12 shows a specific example of history information.

FIG. 13 shows an example of a display screen.

FIG. 14 shows an example of a display screen.

### DESCRIPTION OF EXEMPLARY EMBODIMENTS

Hereinafter, embodiments will be described. Note that the embodiments described below are not intended to unduly limit the content of the invention described in the appended claims. Further, all the configurations described in the embodiments are not necessarily essential components of the invention.

### 1. Method According to Embodiment

First, a method according to this embodiment will be described. In the activity at a high altitude such as mountain climbing, a person goes into a hypoxic state to cause symptoms of altitude sickness (high altitude disorder) in some cases. Specifically, symptoms such as headache, nausea, and sleep disorder are considered, and sometimes a serious symptom such as cerebral edema or pulmonary edema is caused.

Therefore, as disclosed in Patent Document 1, there has been known a method for notifying a user of the risk of an activity of mountain climbing or the like. However, it is difficult to cope with individual differences based only on altitude information, and it is not easy to present appropriate information.

On the other hand, a pulse oximeter which has been widely used for medical and health care application is considered to be utilized in an activity at a high altitude. It is possible to measure an arterial oxygen saturation (SpO₂) by attaching a widely known portable pulse oximeter to a fingertip. For example, a user who climbs up a mountain confirms the user's own condition periodically or when the user feels something strange in the physical condition by attaching a carried pulse oximeter to a fingertip and measuring SpO₂. Then, in the case where SpO₂ has dropped, the user takes measures, for example, the user takes a rest or stops climbing up the mountain and starts climbing down the mountain.

However, the pulse oximeter in the related art does not take into consideration continuous wearing. Specifically, in the measurement of SpO₂ by a method in the related art, it is necessary to perform the following procedure: the activity is once stopped, the pulse oximeter is taken out of the carried goods and attached to a fingertip, and the user keeps quiet until the measurement is completed. In other words, the user cannot ascertain the user's own SpO₂ state unless the measurement of SpO₂ is actively performed.

Such a method has two problems. Firstly, the measurement of SpO₂ inhibits the activity (exercise) of a user. As described above, a widely known pulse oximeter is of a type such that it is attached to a fingertip and does not take into consideration an activity while attaching the device thereto, and therefore, the activity such as mountain climbing is once stopped, and then, the measurement of SpO₂ should be performed. Further, it is difficult to catch something using the finger used for the measurement during the measurement. In the case where an attachment section (a measurement unit and a sensor section) to a fingertip and a main body section (a processing section and a display section) of the pulse oximeter are separated bodies, a cable which connects the attachment section to the main body section becomes an obstacle, and therefore, the activity of the user is inhibited.

Secondly, there is a time lag from when the user goes into a hypoxic state until when symptoms of altitude sickness are actually developed, and therefore, it is difficult to prevent the symptoms of altitude sickness. For example, even in the case of headache which is a relatively light symptom, it is said that it takes several hours from when a person goes into a hypoxic state until when the person feels headache. Further, in the case of a serious symptom such as cerebral edema or pulmonary edema, it takes a time in a unit of several days from when a person goes into a hypoxic state until when the person develops the symptom. That is, it is too late even if SpO₂ is measured after the user is aware of ill health, and it is not useful for prevention of the symptoms of altitude sickness. However, when SpO₂ is tried to be measured frequently, the activity such as mountain climbing is inhibited each time, and therefore, the frequent measurement is not preferred.

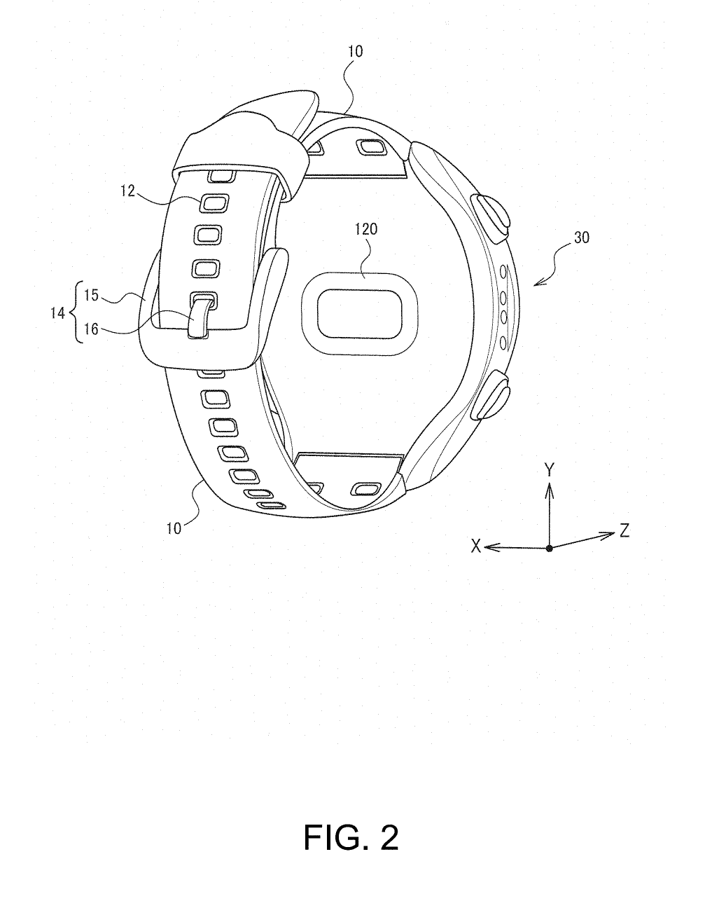

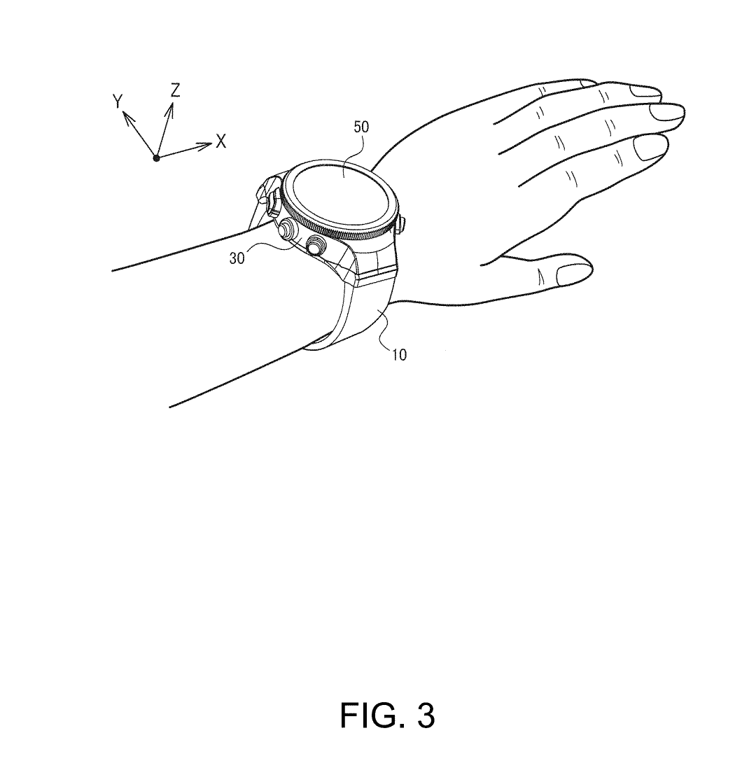

In view of this, the present applicant proposes a method for acquiring arterial oxygen saturation information (information associated with SpO₂) in a wearable device. The wearable device is a device enabling continuous wearing on a part of the body of a user, and may be, for example, a watch-type device as described later with reference to FIGS. 2 and 3. Alternatively, the wearable device is a wrist-type device, and the attachment site may be an ankle, an upper arm, or the like. According to this configuration, SpO₂ can be measured frequently (in a narrow sense, continuously) without inhibiting the activity of a user, and therefore, the above-mentioned two problems can be solved. For example, it becomes possible to contribute to the prevention of the symptoms of altitude sickness by displaying the value of continuously measured SpO₂, or by giving an alert when the value of SpO₂ becomes a dangerous value.

However, it cannot be said to be sufficient even if SpO₂ is continuously measured by a wearable device and the results are notified. It is because even in the case of a healthy user, the value of SpO₂ changes depending on the environment. For example, as will be described later with reference to FIG. 7, a standard value of SpO₂ changes as the altitude is higher. In the case of FIG. 7, when the altitude is about 3000 m, even in the case of a healthy user, SpO₂ can decrease to about 87(%). Even in the same situation that SpO₂ is 87, if the measurement site is 0 m above the sea level or at an altitude close to 0 m, some kind of abnormality in the vascular system is suspected. However, if the altitude is 3000 m, acclimatization to a high altitude is appropriately achieved, and the risk of development of symptoms of altitude sickness is considered to be low. That is, it is difficult to make an appropriate notification by merely considering only SpO₂. In addition, environmental information other than the altitude, or the personal data or biological information other than SpO₂ of a user also has a correlation with the value of SpO₂ in some cases.

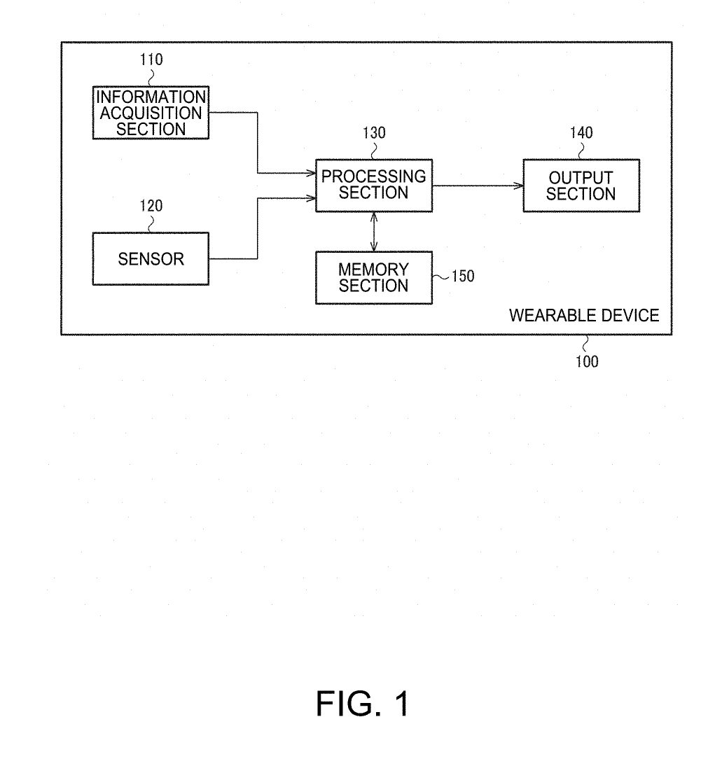

In view of this, the present applicant proposes a method for generating and outputting notification information capable of giving an appropriate notification to a user. As shown in FIG. 1, a wearable device 100 according to this embodiment includes an information acquisition section 110 which acquires environmental information including at least one of location information of a user, atmospheric pressure information, and weather information, a sensor 120 which acquires arterial oxygen saturation information, a processing section 130 which generates notification information associated with an arterial oxygen saturation based on the environmental information and the arterial oxygen saturation information, and an output section 140 which outputs the notification information.

Here, the location information of a user is information indicating the location of a user. The location as used herein includes a location in a horizontal direction and a direction in a vertical direction. The location in a horizontal direction corresponds to a latitude and a longitude, and the location in a vertical direction corresponds to an altitude. The atmospheric pressure information is information associated with an air pressure around a user (atmospheric pressure). The atmospheric pressure information is, for example, the value of an atmospheric pressure or the value of an oxygen partial pressure, and hPa, atm, or the like can be used as the unit. The weather information is information indicating weather (climate) around a user and includes information such as “sunny”, “cloudy”, “rainy”, and “snowy” weather. The weather information is not limited to information indicating the current weather, and may include past weather history and future weather forecast.

The arterial oxygen saturation information is information associated with the arterial oxygen saturation of a user. The arterial oxygen saturation information may be the value of SpO₂ itself, which is a percutaneous arterial oxygen saturation, but is not limited thereto, and may include information for calculating SpO₂ or may be information obtained from SpO₂.

Further, the notification information is information for giving some kind of notification to a user, and is particularly information for making a notification associated with the arterial oxygen saturation in this embodiment. The notification information may be at least one of advice information associated with the arterial oxygen saturation and alert information associated with the arterial oxygen saturation. The advice information as used herein is information for presenting desired action or measures for a user, and is information for making a notification of “optimum pace”, “slow down”, “take rest”, “keep quiet”, etc. described later. Further, the alert information is information for giving a notification of danger to a user, and is, for example, information for displaying a letter string such as “danger”, displaying an image showing danger, changing the background color of the screen, blinking the screen, or generating a light, a vibration or a sound.

According to this configuration, it becomes possible to use the wearable device 100 for generating and outputting notification information, and also becomes possible to use not only arterial oxygen saturation information, but also environmental information for generating notification information. The advantage of using the wearable device 100 is as described above. Further, since environmental information is used, it becomes possible to generate more appropriate notification information in accordance with the environment of a user.

As described above, a standard value (a value of a healthy subject) of SpO₂ changes depending on the altitude. That is, by generating notification information using location information and arterial oxygen saturation information, it becomes possible to generate appropriate notification information in consideration also of the change in SpO₂ in accordance with the location.

Further, the atmospheric pressure changes depending on the weather even if the altitude is the same. In general, when it is sunny, the atmospheric pressure is high, and when it is rainy, the atmospheric pressure is low. Whether or not a user goes into a hypoxic state is associated with an oxygen concentration around the user, and is more specifically associated with an oxygen partial pressure. Assuming that the ratio of oxygen in the atmosphere is constant, the oxygen partial pressure is proportional to the atmospheric pressure. That is, a standard value of SpO₂ also changes depending on the weather, and therefore, by generating notification information using weather information and arterial oxygen saturation information, it becomes possible to generate more appropriate notification information.

Further, as evident from the above description, whether or not a user goes into a hypoxic state is directly associated with atmospheric pressure information which is an atmospheric pressure or an oxygen partial pressure around the user. Therefore, by generating notification information using atmospheric pressure information and arterial oxygen saturation information, it becomes possible to generate more appropriate notification information.

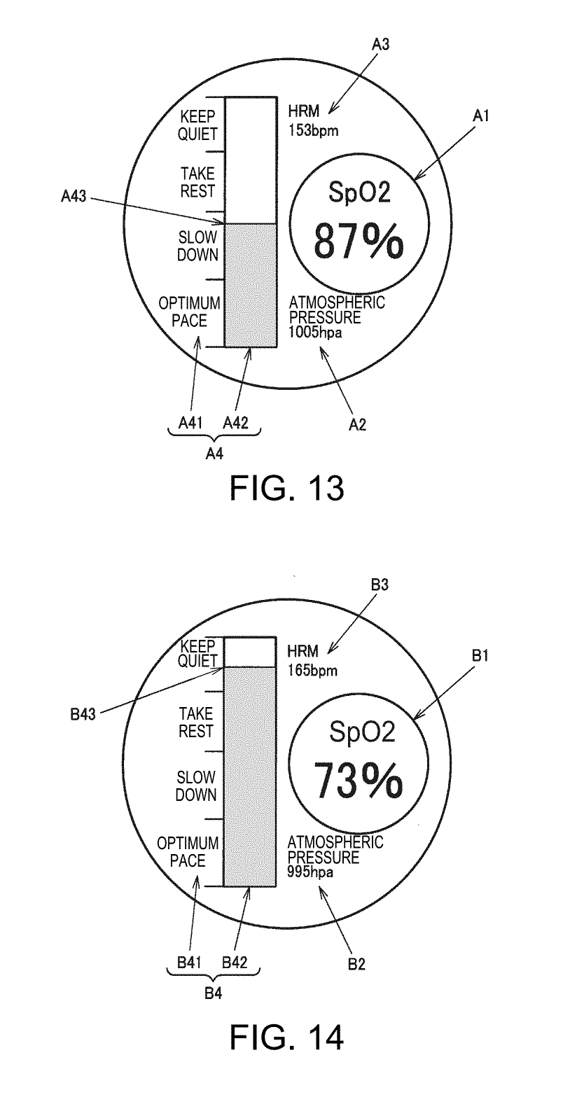

Hereinafter, a configuration example of the wearable device 100 according to this embodiment will be described, and thereafter, a specific example of the notification information generation processing will be described. Incidentally, the respective pieces of information of arterial oxygen saturation information, location information, atmospheric pressure information, and weather information and a detailed method for acquiring (method for generating) other information to be used in this embodiment will be described later along with the notification information generation processing. Finally, an example of the display screen on which notification information to be generated in this embodiment will be described with reference to FIGS. 13 and 14.

### 2. System Configuration Example

A system configuration example of the wearable device 100 is as shown in FIG. 1. The wearable device 100 includes an information acquisition section 110, a sensor 120, a processing section 130, an output section 140, and a memory section 150. However, the wearable device 100 is not limited to the configuration shown in FIG. 1, and various modifications can be made, for example, some constituent elements thereof may be omitted, or other constituent elements may be added, etc.

The information acquisition section 110 acquires at least environmental information. In the case where the wearable device 100 includes a location sensor (for example, a GPS (Global Positioning System) receiver) which acquires location information, or an atmospheric pressure sensor which acquires atmospheric pressure information, the information acquisition section 110 may be realized as an interface for acquiring sensor information, for example, an A/D (Analog-to-Digital) converter which performs A/D conversion of an analog signal from the location sensor or the atmospheric pressure sensor, or as an amplifier which performs amplification processing. Alternatively, in the case where environmental information is acquired by an external device, the information acquisition section 110 is realized as a reception processing section which receives environmental information from the external device.

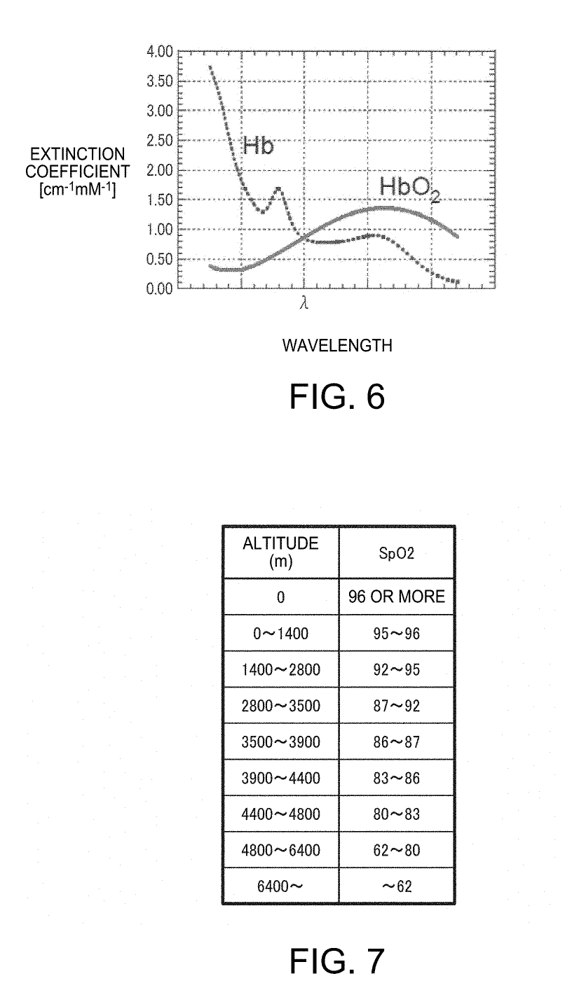

The sensor 120 is a sensor for acquiring at least arterial oxygen saturation information and can be realized by, for example, a photoelectric sensor. The sensor 120 includes a light-emitting section which emits lights with at least two mutually different wavelengths, and a light-receiving section which receives a transmitted light which is a light from the light-emitting section and is transmitted through a test subject or a reflected light which is a light from the light-emitting section and is reflected from a test subject. As one example, the sensor 120 includes a first light-emitting section which emits a light with a first wavelength, a second light-emitting section which emits a light with a second wavelength, a first light-receiving section which receives a transmitted light or a reflected light which is a light from the first light-emitting section and is transmitted or reflected in a living body, and a second light-receiving section which receives a transmitted light or a reflected light which is a light from the second light-emitting section and is transmitted or reflected in the living body. The first wavelength is, for example, a wavelength corresponding to an infrared light, and the second wavelength is a wavelength corresponding to a red light. However, the configuration of the sensor 120 is not limited thereto, and various modifications can be made. For example, instead of providing two light-receiving sections, one light-receiving section may be used in a time-division manner. Alternatively, the lights with the first wavelength and the second wavelength are not limited to the infrared light and the red light, and lights with other wavelengths may be used. A method for obtaining arterial oxygen saturation information based on the output of the sensor 120 will be described later with reference to FIG. 6. Further, the sensor 120 may also functions as a sensor which detects other biological information, in a narrow sense, pulse wave information such as a pulse rate, which will be described in detail later.

The processing section 130 performs various types of processing including notification information generation processing based on environmental information acquired by the information acquisition section 110, and arterial oxygen saturation information from the sensor 120. The function of this processing section 130 can be realized by any of various types of processors such as a CPU (Central Processing Unit), hardware such as an ASIC (Application Specific Integrated Circuit, a gate array, etc.), a program, or the like.

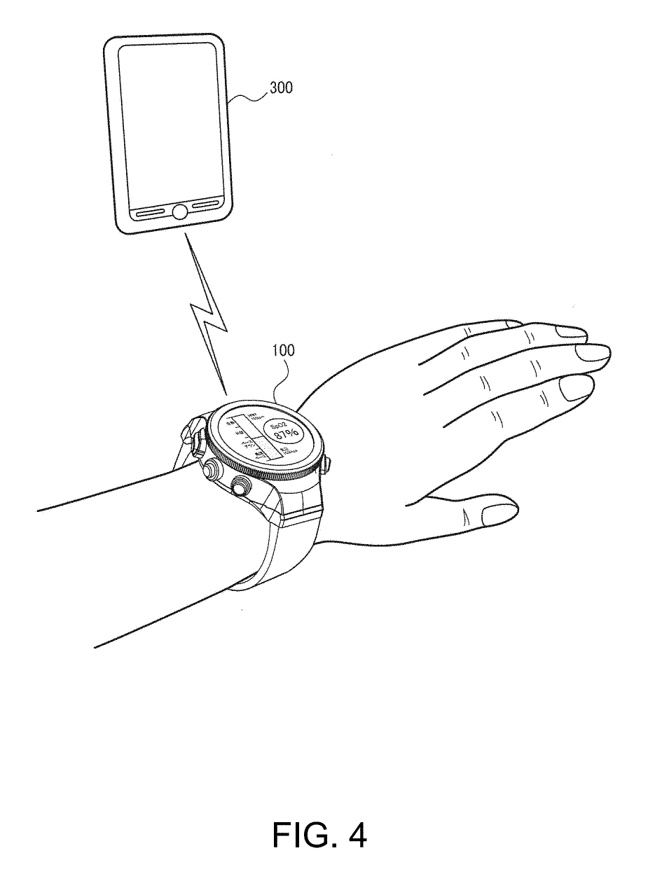

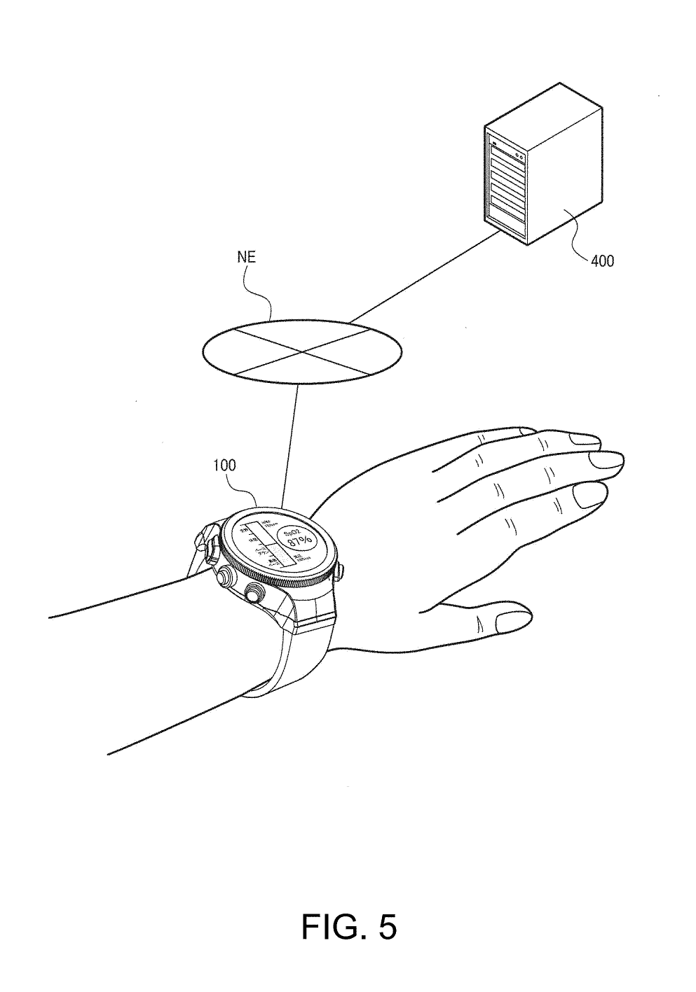

The output section 140 performs the output of notification information generated by the processing section 130. The output section 140 can be realized by various forms, which will be described in detail later along with FIGS. 4 and 5.

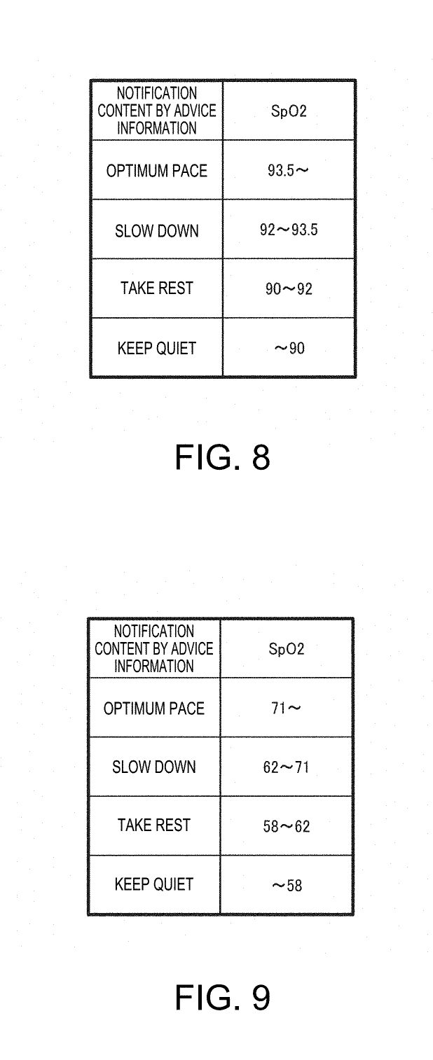

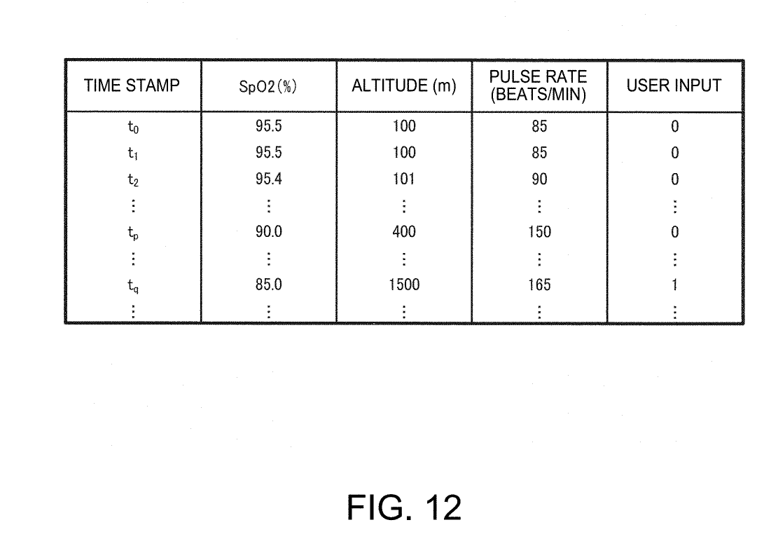

The memory section 150 is used as a work region of the processing section 130 or the like, and its function can be realized by a memory such as an RAM (Random Access Memory), an HDD (Hard Disk Drive), or the like. The memory section 150 may store a table for generating notification information as described later with reference to FIG. 8 or the like, or may store history information of arterial oxygen saturation information or the like as described later with reference to FIG. 12.

FIG. 2 shows an example of an external appearance view of the wearable device 100 according to this embodiment. As shown in FIG. 2, the wearable device 100 includes a case section 30, and a band section 10 for fixing the case section 30 to a part (in a narrow sense, the wrist) of a user, and in the band section 10, a fitting hole 12 and a buckle 14 are provided. The buckle 14 is constituted by a buckle frame 15 and a locking section (projection rod) 16.

FIG. 2 is a perspective view when the wearable device 100 in a state where the band section 10 is fixed using the fitting hole 12 and the locking section 16 is viewed from the direction on a band section 10 side (a surface side which becomes a test subject side in an attached state among the surfaces of the case section 30). In the wearable device 100 shown in FIG. 2, a plurality of fitting holes 12 are provided in the band section 10, and by inserting the locking section 16 of the buckle 14 into any of the plurality of fitting holes 12, the wearable device 100 is attached to a user. The plurality of fitting holes 12 are provided along the longitudinal direction of the band section 10 as shown in FIG. 2.

In the case section 30 of the wearable device 100, the sensor 120 is provided. Here, it is assumed that the sensor 120 is a photoelectric sensor including a light-emitting section and a light-receiving section, and therefore, the sensor 120 is provided at a place where it is exposed to the outside of the case section 30, particularly, on a surface coming into contact with a test subject side in an attached state. That is, in a state where the wearable device 100 is attached, the sensor 120 is closely adhered to the living body, and therefore, the incidence of an external light onto the light-receiving section can be suppressed, or the intensity of a detection signal of the sensor 120 can be increased by decreasing the distance between the sensor 120 and the living body so as to decrease the optical path length.

FIG. 3 is a view when the wearable device 100 in a state where a user wears the device is viewed from a side where the display section 50 is provided. As evident from FIG. 3, the wearable device 100 according to this embodiment includes the display section 50 at a place corresponding to a dial plate of a common watch. In a state of attaching the wearable device 100, a surface on which the sensor 120 is provided in the case section 30 is closely adhered to a test subject, and also the display section 50 provided on a surface on the opposite side to the sensor 120 in the case section 30 is located at a place where a user can easily visually recognize the display section 50.

Incidentally, in FIGS. 2 and 3, a coordinate system is set by using the case section 30 of the wearable device 100 as a base, and a direction which is a direction intersecting the display section 50 and is directed to the front surface from the back surface when the surface on the display surface side of the display section 50 is defined as the front surface is defined as “Z-axis positive direction”. In a state where the wearable device 100 is attached to a test subject, the above-mentioned Z-axis positive direction corresponds to a direction directed from the test subject to the case section 30. Further, two axes orthogonal to the Z-axis are defined as X-axis and Y-axis, and particularly, a direction on which the band section 10 is attached to the case section 30 is set to the Y-axis.

The notification information generated by the processing section 130 may be used for giving a notification to a user in the wearable device 100. For example, the output section 140 is the display section 50 provided in the wearable device 100, and the display section 50 may display notification information. In this case, the display section 50 which is the output section 140 may display at least one of advice information associated with the arterial oxygen saturation and alert information associated with the arterial oxygen saturation as the notification information. A specific example of the display screen will be described later with reference to FIGS. 13 and 14.

The notification in the wearable device 100 is not limited to the display. For example, the output section 140 may be realized by a light-emitting section, a vibration generation section, a speaker, or the like provided in the wearable device 100. In this case, as the output form of the notification information, the output is performed by generation of a light, a vibration, or a sound.

The notification to a user using the notification information may be made by a device which is different from the wearable device 100, and in this case, this is realized as a communication section (transmission processing section) which transmits the notification information to a device which is different from the output section 140.

FIG. 4 shows an example of connection between the wearable device 100 and another device. As shown in FIG. 4, the wearable device 100 may be connected to a portable terminal device 300 such as a smartphone through a short-distance radio communication. The portable terminal device 300 is carried by a user who performs an activity such as mountain climbing, and therefore, the notification to the user using the notification information may be performed by the wearable device 100 or may be performed by the portable terminal device 300 or may be performed by both the devices. In FIG. 4 and the below-mentioned FIG. 5, an example in which the wearable device 100 includes the display section 50 and the notification information is displayed on the display section 50 is shown, however, this configuration is not essential.

In the case where the notification is made by the portable terminal device 300, the output section 140 of the wearable device 100 is realized as a communication section (transmission processing section) which transmits notification information. The portable terminal device 300 receives notification information from the wearable device 100 by a communication section (transmission processing section) of the portable terminal device 300 and makes a notification based on the received notification information. For example, the notification may be displayed by a display section of the portable terminal device 300, or may be made using a light, a sound, a vibration, or the like.

The device in which the notification using the notification information is made is not limited to a device which is carried by a user who performs an activity such as mountain climbing. FIG. 5 shows another example of connection between the wearable device 100 and another device. As shown in FIG. 5, the wearable device 100 may be connected to an information processing device 400 such as a PC (Personal Computer) through a network NE. As the network NE here, various networks such as a WAN (Wide Area Network), a LAN (Local Area Network), and a short-distance radio communication can be used. Further, the information processing device 400 in FIG. 5 may be a portable terminal device which is carried by a user who is different from a user who performs an activity such as mountain climbing.

In the case where the notification is made by the information processing device 400, the output section 140 of the wearable device 100 is realized as a communication section (transmission processing section) which transmits notification information. Specific notification by the information processing device 400 can be realized by any of various methods in the same manner as in the case of the portable terminal device 300.

In the case of the example in FIG. 5, the user to whom the notification is given by the information processing device 400 is a user who is different from a user who performs an activity such as mountain climbing. According to this, a change in the SpO₂ of a person who uses the wearable device 100 and performs an activity at a high altitude or a change in another index is observed, it is possible to notify a person who is in a different place such as on the level ground of the condition of the person who uses the device through the network NE. For example, it is possible to notify the family of the person who uses the device of the current value or history of SpO₂ or a pulse rate, or whether the person who uses the device moves, or the like, and sometimes, it is possible to notify another user of the danger of the person who uses the device. That is, in this case, the communication section which is the output section 140 of the wearable device 100 transmits alert information associated with the arterial oxygen saturation as the notification information. Further, the user who receives the notification based on the notification information is not limited to the family of the person who uses the wearable device 100, and may be a director of a mountain lodge, a member of the mountain climbing group, etc.

Further, it is also possible to realize the processing of the respective sections of the wearable device 100 according to this embodiment by a program. That is, the method of this embodiment can be applied to a program which causes a computer to execute acquiring environmental information including at least one of location information of a user, atmospheric pressure information, and weather information, generating notification information associated with an arterial oxygen saturation based on arterial oxygen saturation information from a sensor and the environmental information, and outputting the notification information.

Further, the wearable device 100 of this embodiment includes a memory which stores information (for example, a program or various types of data), a processor which operates based on the information stored in the memory, and the sensor 120. The processor performs information acquisition processing for acquiring environmental information including at least one of location information of a user, atmospheric pressure information, and weather information, notification information generation processing for generating notification information associated with an arterial oxygen saturation based on the environmental information and arterial oxygen saturation information from the sensor 120, and output processing for outputting the notification information.

The processor may be realized by, for example, hardware in which the functions of the respective sections are separated individually, or may be realized by hardware in which the functions of the respective sections are integrated. The processor may be, for example, a CPU. However, the processor is not limited to a CPU, and various types of processors such as a GPU (Graphics Processing Unit) and a DSP (Digital Signal Processor) can be used. Further, the processor may be a hardware circuit by an ASIC. The memory may be, for example, a semiconductor memory such as an SRAM (Static Random Access Memory) or a DRAM (Dynamic Random Access Memory), may be a register, may be a magnetic memory device such as a hard disk device, or may be an optical memory device such as an optical disc device. For example, the memory stores a computer-readable instruction, and by executing the instruction by the processor, the function of each section of an image processing device is realized. The instruction as used herein may be an instruction of an instruction set constituting the program, or may be an instruction for instructing the hardware circuit of the processor on the operation.

The operation of this embodiment is realized, for example, as follows. The processor acquires environmental information and arterial oxygen saturation information and stores the information in the memory. The processing for acquiring the environmental information may be realized by acquiring information from the sensor 120, a location sensor such as a GPS receiver, or an atmospheric pressure sensor by the processor, or may be realized by receiving information from an external device by the processor. Then, the processor reads out the environmental information and the arterial oxygen saturation information from the memory, generates notification information associated with the arterial oxygen saturation based on the two pieces of information, and stores the notification information in the memory. Further, the processor reads out the notification information from the memory and performs output processing of the notification information.

Each section of the wearable device 100 of this embodiment is realized as a module of the program which operates on the processor. For example, the information acquisition section 110 is realized as an information acquisition module which acquires environmental information including at least one of location information of a user, atmospheric pressure information, and weather information. The processing section 130 is realized as a processing module which generates notification information associated with an arterial oxygen saturation based on the environmental information and the arterial oxygen saturation information. The output section 140 is realized as an output module which outputs the notification information.

### 3. Notification Information Generation Processing

Next, the processing for generating the notification information using the arterial oxygen saturation information and other information will be described in detail. The “other information” as used herein may be environmental information as described above, or may be biological information other than SpO₂ such as a pulse rate, or may be the personal data of a user such as age and gender.

Hereinafter, first, a method for acquiring various types of information (calculation method) will be described, and thereafter, a specific method for notification information generation processing using the information will be described. Further, a modification example in which history information is used for generating the notification information will also be described.

### 3.1 Method for Acquiring Various Types of Information Arterial Oxygen Saturation Information

FIG. 6 is a view showing the optical absorption spectrum of reduced hemoglobin Hb and the optical absorption spectrum of oxyhemoglobin HbO₂. As shown in FIG. 6, the optical absorption spectra of oxyhemoglobin HbO₂ and reduced hemoglobin Hb are different, and in the case where a light with a relatively long wavelength λ1 (>λ) is irradiated, the extinction coefficient for the light of oxyhemoglobin HbO₂ is larger than that of reduced hemoglobin Hb, and therefore, the intensity of a transmitted light or a reflected light (the output value of the light-receiving section: V1) of the light in a living body serves as an index value indicating the amount of oxyhemoglobin in the blood vessel. Similarly, in the case where a light with a relatively short wavelength λ2 (<λ) is irradiated, the extinction coefficient for the light of reduced hemoglobin Hb is larger than that of oxyhemoglobin HbO₂, and therefore, the intensity of a transmitted light or a reflected light (the output value of the light-receiving section: V2) of the light in a living body serves as an index value indicating the amount of reduced hemoglobin in the blood vessel. Due to this, V1/(V1+V2) serves as an index value indicating the ratio of oxyhemoglobin, that is, a value having a correlation with the arterial oxygen saturation SpO₂.

In this manner, by utilizing the characteristic that the optical absorption spectra of oxyhemoglobin HbO₂ and reduced hemoglobin Hb are different, it is possible to obtain arterial oxygen saturation information from transmitted lights or reflected lights of lights with two different wavelengths. In the above description, a simple method is explained, however, various modifications of the method for obtaining SpO₂ or information associated with SpO₂ using an infrared light and a red light are known, and in this embodiment, these can be widely applied. In addition, the wavelengths to be used are not limited to the infrared light and the red light, and may be any as long as the absorbances of oxyhemoglobin HbO₂ and reduced hemoglobin Hb for one wavelength are clearly different from the absorbances for the other wavelength. For example, a modification in which one light is changed to a green light can be made.

Hereinafter, a description will be made by assuming that the value of SpO₂ (unit: %) is determined based on information from the sensor 120 in the processing section 130, however, a modification in which the form of the arterial oxygen saturation information is different can be made.

### Environmental Information (Location Information)

In the wearable device 100 (in a narrow sense, the case section 30) according to this embodiment, a GPS receiver may be provided, and acquisition of location information is performed based on a GPS signal. Alternatively, as shown in FIG. 4, in the case where the portable terminal device 300 carried by a user and the wearable device 100 operate interlockingly, the portable terminal device 300 includes a GPS receiver, and the wearable device 100 may acquire a GPS signal from the portable terminal device 300.

It is possible to acquire a latitude, a longitude, and a height of a user from the GPS signal, and the location information of this embodiment is at least one of them. The height (GPS height) acquired directly from the GPS signal does not strictly match an altitude which is a height above the sea level. The altitude according to this embodiment may be information obtained by correction processing of the GPS height, or the GPS height may be used in place of the altitude. Alternatively, a height corresponding to the current latitude and longitude may be determined as the altitude based on latitude and longitude information and map information.

Further, an altitude with high accuracy may be determined based on the GPS signal and the information from the atmospheric pressure sensor. It is known that the atmospheric pressure and the height have a correlation, and the value of one of them can be calculated from the value of the other. Therefore, in this embodiment, an altitude with high accuracy may be determined by performing correction processing using both of the height determined from the atmospheric pressure (hereinafter referred to as “atmospheric pressure height”) and the GPS height.

However, various modifications can be made to the acquisition of the location information. For example, there is a possible case where a GPS signal is blocked by a high mountain and cannot be acquired. In this case, the location may be estimated using the output from, for example, an acceleration sensor or a gyro sensor. The estimation of the location using an acceleration sensor or a gyro sensor is a widely known method as dead reckoning, and therefore, a detailed description will be omitted.

Further, in the case where a GPS signal cannot be acquired, but information can be acquired from the atmospheric pressure sensor, it is possible to utilize the above-mentioned atmospheric pressure height as the altitude. Other than this, various modifications can be made to a specific sensor for obtaining location information.

Further, the location information of this embodiment is not limited to information indicating a location (a location in a horizontal direction or an altitude) at given one timing, and may be information indicating a change in the location in a predetermined time. For example, the degree of increase in the altitude in a unit time is determined as a rising pace, and the rising pace may be used as the location information.

### Environmental Information (Atmospheric Pressure Information)

In the wearable device 100 (in a narrow sense, the case section 30) according to this embodiment, an atmospheric pressure sensor may be provided, and acquisition of atmospheric pressure information is performed based on sensor information from the atmospheric pressure sensor. Alternatively, the portable terminal device 300 may include an atmospheric pressure sensor. The atmospheric pressure sensor is widely known, and therefore, a detailed description will be omitted.

Further, what is directly associated with whether or not a user goes into a hypoxic state is not an atmospheric pressure, but an oxygen partial pressure. Therefore, the processing section 130 of the wearable device 100 determines an oxygen partial pressure based on the atmospheric pressure, and the oxygen partial pressure may be used as the atmospheric pressure information. Alternatively, atmospheric pressure information may be calculated based on the height determined from a GPS signal or the like.

Further, also the atmospheric pressure information is not limited to the information indicating an atmospheric pressure at given one timing, and may be information indicating a change in the atmospheric pressure in a predetermined time.

### Environmental Information (Weather Information)

The weather information includes information associated with weather such as “sunny”, “cloudy”, and “rainy” weather. Such information may be obtained by acquiring weather data presented by an institution such as the Meteorological Agency. That is, the information acquisition section 110 acquires weather information by accessing a server disclosing current weather, past weather, or future weather forecast through a network NE.

Alternatively, a weather forecast may be made based on information of a weather sensor. The weather sensor is a sensor which measures a temperature, a humidity, an illuminance, an ultraviolet radiation dose, a rainfall amount, a wind speed, a wind direction, an atmospheric pressure, etc. The weather sensor can be realized by a widely known weather meter. There is nothing prohibiting the wearable device 100 from including the weather sensor to perform calculation of weather information based on the sensor information in the processing section 130. However, in consideration of a size and a calculation ability, it is natural that the placement of the weather sensor and the weather information calculation processing based on the sensor information are performed in a device which is different from the wearable device 100.

### Biological Information (Pulse Rate)

For the generation of notification information, biological information other than SpO₂ may be utilized, and specifically, pulse wave information such as a pulse rate may be used. A pulse wave appears as a change in the volume of blood, and therefore, a pulse wave sensor measures a pulse wave by capturing a change in the blood flow at a site to become a measurement object. In consideration of the fact that there is a correlation between the blood flow and the amount of hemoglobin in the blood, in the case where the blood vessel is irradiated with a light, when the blood flow is high and also the amount of hemoglobin is large, the amount of light absorption is large and the intensity of a transmitted light or a reflected light is decreased. On the other hand, when the blood flow is low and also the amount of hemoglobin is small, the amount of light absorption is small and the intensity of a transmitted light or a reflected light is increased. That is, it is possible to detect the pulse wave information based on a change over time in a detection signal in a photoelectric sensor.

The light irradiated by a light-emitting section of the pulse wave sensor is preferably a light with a wavelength which is easily absorbed by hemoglobin, and a green light is generally used. Therefore, in the wearable device 100 of this embodiment, the sensor 120 for detecting arterial oxygen saturation information and the pulse wave sensor may be provided separately. In this case, the wearable device 100 includes first and second light-emitting sections which emit a light with a first wavelength (for example, an infrared light) and a light with a second wavelength (a red light), respectively, for arterial oxygen saturation information, and a third light-emitting section which emits a light with a third wavelength (a green light) for pulse wave information. As the light-receiving sections, first to third light-receiving sections may be provided so as to correspond to the first to third light-emitting sections, or one or two light-receiving sections may be provided and used in a time-division manner.

Alternatively, the pulse wave information may be detected using the sensor 120 for detecting arterial oxygen saturation information. Specifically, any of the first light-emitting section and the second light-emitting section included in the sensor 120 is used also as the light-emitting section for detecting pulse wave information. In this case, two light wavelengths are used, and therefore, the pulse wave information is detected using a red light, and the arterial oxygen saturation information is detected using an infrared light and a red light. Alternatively, the pulse wave information is detected using a green light, and the arterial oxygen saturation information is detected using a red light and a green light. Other than these, various modifications can be made to the wavelength of the light.

Further, the pulse wave information is not limited to the pulse rate, and may be a pulse interval (PR interval), or may be a variation in pulse interval, or may be other information indicating a pulse wave. Hereinafter, an example in which the processing section 130 determines the pulse rate (beats/min, bpm) as the pulse wave information will be described.

### Personal Data

Further, for the generation of the notification information, the personal data of a user may be utilized. The personal data as used herein is information such as age, gender, height, body weight, etc. of a user. The personal data is, for example, input (set) at the time of initial setting by a user. As one example, an initial setting screen is displayed on the display section 50 of the wearable device 100, and the user is encouraged to input the personal data. The user inputs the personal data such as age using an operation interface provided in the wearable device 100 according to the initial setting screen. The operation interface as used herein is, for example, a button provided on the side surface of the case section 30 as shown in FIG. 2 or 3, but is not limited thereto. For example, the display section 50 is realized by a touch panel, and the touch panel may be used as the operation interface.

### 3.2 Utilization of Environmental Information and Biological Information

Next, the notification information generation processing using the arterial oxygen saturation information and other information will be described in detail. As described above, in this embodiment, the notification of information associated with a user who performs an activity at a high altitude such as mountain climbing is assumed. In consideration of the health and safety of the user, as the notification information, advice information associated with the arterial oxygen saturation may be used.

More specifically, the advice information is advice information associated with the continuation of the exercise or the load of the exercise of the user. That is, advice information such that whether or not the user should continue the exercise such as mountain climbing, or whether the load of the exercise can be increased, or whether the load of the exercise should be decreased is generated.

Although it is understood that the risk is higher as the arterial oxygen saturation is lower and the risk is lower as the arterial oxygen saturation is higher, it is not easily understood what action should be taken in a case with a particular value specifically. In that regard, according to the method of this embodiment, a specific advice can be given on whether or not the user can continue the exercise or whether the load of the exercise is high or low, and therefore, an appropriate notification in accordance with the state of the user can be realized.

For example, the processing section 130 determines which range of a first range to an N-th (N is an integer of 2 or more) range the arterial oxygen saturation falls in based on the arterial oxygen saturation information, and when the arterial oxygen saturation is determined to fall in an i-th (i is an integer that satisfies the following relationship: 1≦i≦N) range, the processing section 130 generates i-th advice information.

According to this, a plurality of pieces of advice information for which the contents of advice to be made for the user are different are prepared beforehand, and each piece of advice information can be made to correspond to the numerical range of SpO₂. The contents of advice (the continuation of the exercise, the load of the exercise, etc.) to be made based on a plurality of pieces of advice information are, for example, “optimum pace”, “slow down”, “take rest”, and “keep quiet”, and in this case, N=4.

The advice of “optimum pace” corresponds to a case where the arterial oxygen saturation is in a relatively high numerical range, and indicates that there is no problem in the current arterial oxygen saturation of the user and the exercise is performed at an appropriate pace. That is, the advice of “optimum pace” is information for advising the user to continue the exercise at the current exercise load. Alternatively, in the case where the advice is “optimum pace”, the user can afford to increase the pace, and therefore, some users try to increase the pace until the advice of “slow down” or the like is given in some cases. That is, the advice of “optimum pace” includes an aspect of information for advising the user to continue the exercise at a higher exercise load than the current load.

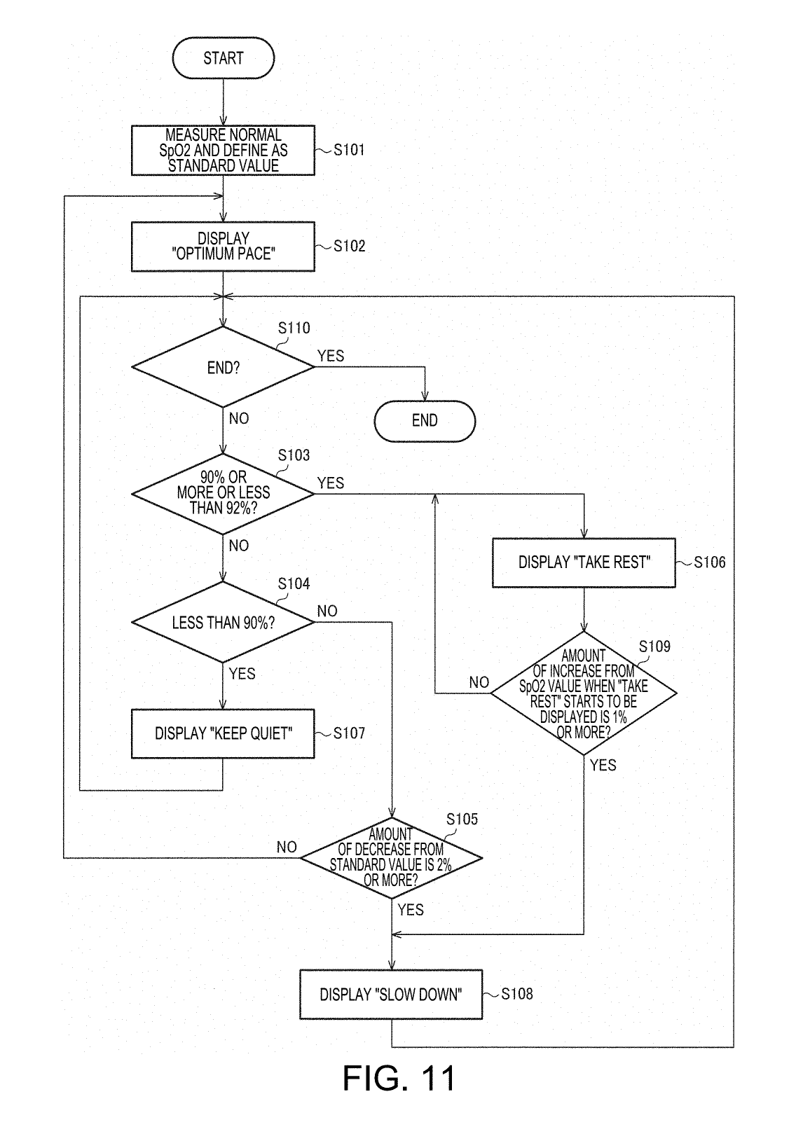

The advice of “slow down” corresponds to a case where the arterial oxygen saturation is in the numerical range which is lower than in the case of “optimum pace” and higher than in the case of “take rest”, and indicates that a problem began to occur in the arterial oxygen saturation of the user, and it is not necessary to take a rest, but the exercise is performed at a too high pace. That is, the advice of “slow down” is information for advising the user to continue the exercise at a lower exercise load than the current load. As will be described later with reference to FIG. 11, it is considered that a state where “take rest” or “keep quiet” is displayed, that is, a state where an exercise is not performed shifts to a state where “slow down” is displayed. That is, the advice of “slow down” here is not limited to information for advising the user to decrease the exercise intensity as compared with the current exercise, and may be information for advising the user to decrease the exercise intensity as compared with the standard exercise for the user.

The advice of “take rest” corresponds to a case where the arterial oxygen saturation is in the numerical range which is lower than in the case of “slow down” and higher than in the case of “keep quiet”, and indicates that the problem of the arterial oxygen saturation of the user is increased, and it is not preferred to continue the exercise. That is, the advice of “take rest” is information for advising the user not to continue the exercise.

The advice of “keep quiet” corresponds to a case where the arterial oxygen saturation is in the numerical range which is higher than in the case of “take rest”, and indicates that the problem of the arterial oxygen saturation of the user is very large, and the motion is suppressed as much as possible so as to recover the condition. That is, the advice of “keep quiet” is information for advising the user not to continue the exercise, and moreover, also not to continue a light activity.

According to this, which advice information is generated and notified among a plurality of pieces of advice information set in a stepwise manner can be determined based on the arterial oxygen saturation (the value of SpO₂), and it is possible to generate appropriate advice information according to the SpO₂ of the user. Incidentally, an example of the above-mentioned four steps is shown here, however, those skilled in the art can easily understand that various modifications can be made to specific setting of the advice information.

In this manner, in the case where a plurality of pieces of advice information and the range of SpO₂ are made to correspond to each other, it is easy to specify the magnitude of the numerical value among the plurality of pieces of advice information. In the case of the above-mentioned example, for example, the numerical value of SpO₂ has the following magnitude relationship: “slow down”>“take rest”, and more specifically, the following relationship: the lower limit of the range of “slow down”>the upper limit of the range of “take rest” may be used.

However, it is not preferred to set the ranges corresponding to the respective pieces of advice information to fixed values. As described above, the value of SpO₂ changes according to the environmental information of a user, and even in a case where the value of SpO₂ falls in the given range, there is no health risk and the advice may be determined as “optimum pace” in some cases, or the risk is very high, and the advice should be determined as “keep quiet” in some cases.

Therefore, the processing section 130 may change the first range to the N-th range described above according to the environmental information. More specifically, the processing section 130 changes the arterial oxygen saturation corresponding to at least one range of the first range to the N-th range according to the environmental information. FIG. 7 is a view showing a relationship between the value of SpO₂ of a healthy subject and the altitude. In FIG. 7, the description of “α-β” indicates a or more and less than β, and the same shall also apply to FIG. 8 and FIG. 9. As evident from FIG. 7, even in the case of a healthy subject, as the altitude is higher, the value of SpO₂ is decreased. For example, when the altitude is 0 m, the SpO₂ of a healthy subject is 96% or more, and in the case where the SpO₂ is lower than 90%, some kind of disease is suspected. However, in the case where the altitude is 2800 m or higher, it is general that the even in the case of a healthy subject, the value of SpO₂ is lower than 90%. Due to this, in the case where the altitude is 2800 m or higher, even if the SpO₂ is 89% or the like, the necessity of generation of advice information of “take rest” or “keep quiet” or generation of alert information for notifying of danger is low.

That is, in this embodiment, the ranges of SpO₂ to be made to correspond to “optimum pace” “slowdown”, “take rest”, and “keep quiet”, respectively, are changed according to the altitude. FIG. 8 is a view showing an example of the ranges of SpO₂ corresponding to the respective pieces of advice information in the case where the altitude is 2000 m. As shown in FIG. 7, in the case where the altitude is 2000 m, the SpO₂ of a healthy subject is 92 or more and less than 95, and therefore, by using the numerical values of 92 or more and less than 95 as a standard, the numerical ranges corresponding to the respective pieces of advice information are set.

In the example in FIG. 8, a range of 92 or more, which is a normal range, is divided into two, and a range of 92 or more and less than 93.5 is defined as “slow down”, and a range of 93.5 or more is defined as “optimum pace”. It is because the range of 92 or more is the normal range, and therefore, at least the exercise can be continued. On the other hand, in the case where the SpO₂ is less than 92, the SpO₂ is outside the normal range, and therefore, advice is given so as not to continue the exercise. Specifically, a range of 90 or more and less than 92 is defined as “take rest”, and a range of less than 90 is defined as “keep quiet”.

By using the table shown in FIG. 8, it becomes possible to generate appropriate advice information in the case where the altitude is 2000 m. Further, in the case where the altitude is different, with reference to FIG. 7, a table of ranges corresponding to the altitude may be utilized. FIG. 9 is a view showing an example of the ranges of SpO₂ corresponding to the respective pieces of advice information in the case where the altitude is 5000 m. As shown in FIG. 7, in the case where the altitude is 5000 m, the SpO₂ of a healthy subject is 62 or more and less than 80, and therefore, by using the numerical values of 62 or more and less than 80 as a standard, the numerical ranges corresponding to the respective pieces of advice information are set. As evident from comparison between FIG. 8 and FIG. 9, by preparing a plurality of tables corresponding to the altitudes beforehand in this manner, even if the value of SpO₂ is the same, different pieces of advice information can be generated, and it becomes possible to appropriately correspond to the change in the altitude. That is, the processing section 130 changes the arterial oxygen saturation information (the value of SpO₂, and in a narrow sense, at least one of the upper limit and the lower limit of the numerical range) corresponding to the respective ranges from the first range to the N-th range according to the environmental information. However, in FIGS. 8 and 9, an example in which the upper limit and the lower limit of SpO₂ corresponding to all the ranges from the first range to the N-th range are changed is shown, however, the processing of this embodiment is not limited thereto. The processing section 130 may change the arterial oxygen saturation information (the upper limit and the lower limit of SpO₂) corresponding to k ranges (k is an integer of 1 or more and N−1 or less) of the first range to the N-th range according to the environmental information. In the example in FIGS. 8 and 9, an embodiment in which the upper limit and the lower limit of SpO₂ corresponding to “keep quiet” and the lower limit of SpO₂ corresponding to “take rest” are changed, but the upper limit and the lower limit of SpO₂ corresponding to “slow down” and “optimum pace” are not changed can be also adopted.

Further, the table for determining the advice information from SpO₂ is not limited to the configuration in FIGS. 8 and 9. For example, some users have a high normal SpO₂, and some users have a low normal SpO₂. This is because there is a difference in cardiopulmonary function among users, or there is a difference in the altitude of place of residence. In this case, not only merely a high or low value of SpO₂, but also the degree of change from the normal value (standard value) can be used as the index of the load on the user. Specifically, when the current SpO₂ greatly decreases from the standard value, the load is large, and when the degree of decrease is small, the load is small. Therefore, which of the plurality of pieces of advice information is notified may be determined by also taking into account the amount of change from the standard value.

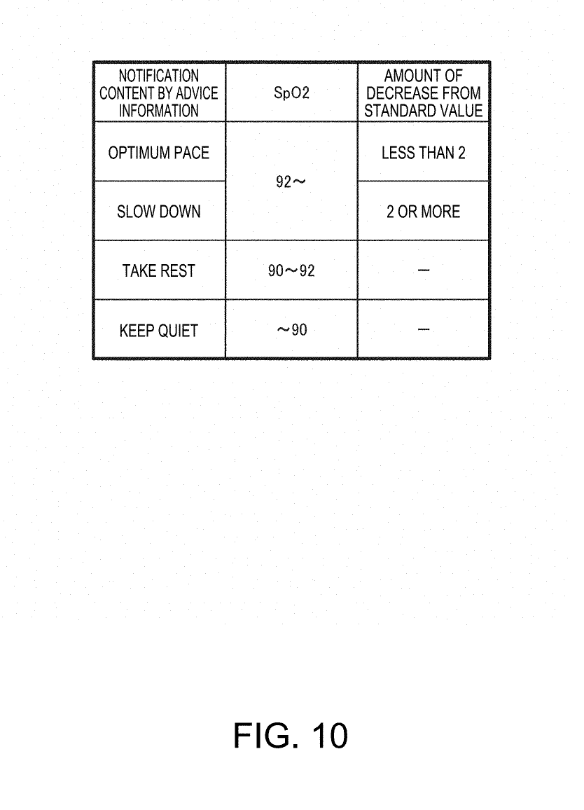

FIG. 10 is another table showing a relationship between the respective pieces of advice information and SpO₂ in the case where the altitude is 2000 m. As described above, in the case where the altitude is 2000 m, the SpO₂ of a healthy subject is 92 or more and less than 95. Therefore, when the current value of SpO₂ is 92 or more, it is determined that at least the exercise can be continued, and the advice is defined as “optimum pace” or “slow down”. Then, it is determined which advice information for making a notification of “optimum pace” or “slow down” is generated according to the degree of decrease from the standard value. The change in question here is a change that the SpO₂ decreases, and therefore, in the example in FIG. 10, a case where the amount of decrease from the standard value is less than 2 is defined as “optimum pace”, and a case where the amount of decrease from the standard value is 2 or more is defined as “slow down”. The advices of “take rest” and “keep quiet” are determined based on the value of SpO₂ in the same manner as in the example in FIG. 8.

Further, it may be determined which of the plurality pieces of advice information is generated based on the changing tendency of SpO₂. For example, in the case where “take rest” is displayed, and a user actually takes a rest so that the risk of the user is decreased (the user is acclimated to a high altitude), the value of SpO₂ is considered to increase. That is, a modification in which advice information to be displayed is changed according to the degree of increase in SpO₂ after displaying “take rest” can be also made. For example, an embodiment in which in the case where the current value of SpO₂ is increased by 1 or more from the value of SpO₂ when displaying “take rest”, the advice information to be displayed is changed from “take rest” to “slow down” is considered.

FIG. 11 is a flowchart illustrating processing in the processing section 130 in the case where the altitude is 2000 m. The processing shown in FIG. 11 is started, for example, when an operation input indicating start is received from a user. Alternatively, the processing shown in FIG. 11 may be started when the processing section 130 detects the start of an exercise of a user based on a GPS signal or the like. After this processing is started, first, a standard value of SpO₂ is acquired (Step S101). Further, as an initial setting for the advice information, “optimum pace” is selected and displayed (Step S102).

In the transition from “optimum pace” to another advice information, a determination whether or not the current value of SpO₂ is 90 or more and less than 92 (Step S103), and a determination whether or not the current value of SpO₂ is less than 90 (Step S104) are performed sequentially. In the case where the result of the determination is “Yes” in Step S103, “take rest” is selected and displayed (Step S106). In the case where the result of the determination is “No” in Step S103 and “Yes” in Step S104, “keep quiet” is selected and displayed (Step S107).

Further, in the case where the result of the determination is “No” in both Step S103 and Step S104, it is determined whether or not the amount of decrease from the standard value of SpO₂ is 2 or more (Step S105). In the case where the result of the determination is “No” in Step S105, the SpO₂ is 92 or more and the amount of decrease is less than 2, and therefore, the process is returned to Step S102, and the advice of “optimum pace” is maintained. In the case where the result of the determination is “Yes” in Step S105, the value of SpO₂ itself is as high as 92 or more, but the amount of decrease from the standard value is 2 or more, and therefore, “slow down” is selected and displayed (Step S108).

Further, in the case where the display shifts to “slow down”, the determinations in Step S103, Step S104, and Step S105 are performed sequentially. As described above, in the case where the result of the determination is “Yes” in Step S103, “take rest” is selected and displayed, and in the case where the result of the determination is “Yes” in Step S104, “keep quiet” is selected and displayed. In the case where the result of the determination is “No” in Step S103 and Step S104, and “No” in Step S105, “optimum pace” is selected and displayed. On the other hand, in the case where the result of the determination is “Yes” in Step S105, the process is returned to Step S108, and the advice of “slow down” is maintained.

Further, in the case where the display shifts to “take rest”, it is determined whether or not the current value of SpO₂ is increased by 1 or more from the value of SpO₂ when the advice of “take rest” starts to be displayed (Step S109). In the case where the result of the determination is “No” in Step S109, that is, in the case where recovery of the user is not sufficient, the process is returned to Step S106, and the advice of “slow down” is maintained. In the case where the result of the determination is “Yes” in Step S109, the user has recovered to some extent, and therefore, the process shifts to Step S108, and “slow down” is selected and displayed.

Further, in the case where the display shifts to “keep quiet”, the determinations in Step S103, Step S104, and Step S105 are performed sequentially. As described above, in the case where the result of the determination is “Yes” in Step S103, “take rest” is selected and displayed, and in the case where the result of the determination is “Yes” in Step S104, the advice of “keep quiet” is maintained. In the case where the result of the determination is “No” in Step S103 and Step S104, and “No” in Step S105, “optimum pace” is selected and displayed. On the other hand, in the case where the result of the determination is “Yes” in Step S105, “slow down” is selected and displayed.

Although, the description is omitted above, the processing section 130 may determine whether or not the processing is terminated as appropriate (Step S110). In the determination in Step S110, it is determined as “Yes” in the case where an input of a termination operation by the user is received or in the case where the termination of the exercise of the user is detected. In the case where the result of the determination is “Yes” in Step S110, the processing is terminated, and in the case where the result of the determination is “No” in Step S110, the processing is continued. In FIG. 11, a termination determination is assumed to be performed before a series of determinations using SpO₂, that is, before Step S103, however, it is not limited thereto. For example, the same determination as in Step S110 may be performed after completion of the selection of advice information (after each of Step S102, S106, S107, and S108). Alternatively, independently of the flowchart in FIG. 11, detection of the operation input of a user or detection of the exercise of a user is performed, and in the case where a termination operation or termination of the exercise is detected, an interrupt is issued, whereby the processing in FIG. 11 may be terminated.

The generation processing of any of a plurality of pieces of advice information as described above can also be regarded as a state machine which executes the transition among the states corresponding to the respective pieces of advice information. In the case of the example in FIG. 11, a state machine including at least the following four states: “optimum pace” “slow down”, “take rest”, and “keep quiet” may be used, and from the following three states: “optimum pace” “slow down”, and “keep quiet”, it is possible to maintain the current state or make a transition to any of the other three states. Further, from the state of “take rest”, it is possible to maintain the current state or make a transition to “slow down”. Further, an embodiment in which each state of “optimum pace” “slow down”, “take rest”, and “keep quiet” is only allowed to make a transition to an adjacent state can be also realized, and those skilled in the art can easily understand that various modifications of this embodiment can be considered.

Further, in FIG. 11, an example of the case where the altitude is 2000 m is shown, however, also in the case of other altitudes, the processing by the processing section 130 can be realized according to a similar flowchart. Specifically, the conditions (determination thresholds) for Steps S103 to S105 and S109 may be changed according to the altitude, and more specifically, the conditions may be set with reference to the table data illustrated with reference to FIGS. 7 to 10.

Further, a method for generating the notification information (particularly, the advice information) based on the altitude and the arterial oxygen saturation information is described above. However, it is also possible to use other information for the generation of the notification information.

For example, instead of using the current value of the altitude, a rising pace (climbing pace) is determined based on the change in the altitude per unit time, and the notification information may be generated based on the rising pace. In the case where the rising pace is high, the exercise intensity is high, and therefore, the load on the user is large, and the SpO₂ is likely to decrease. Therefore, it is considered that as compared with the case where the rising pace is low, the degree of reduction of the load when the exercise is stopped is also larger, and therefore, the degree of recovery of SpO₂ by taking a rest is larger by that amount. That is, even in the case where the SpO₂ is the same, as the rising pace is higher, a margin is left more, and as the rising pace is lower, a margin is left less.

Therefore, as one example, advice information may be generated so as to encourage a user to take a rest earlier as the rising pace is lower. Specifically, in the case where the rising pace is low, as compared with the case where the rising pace is high, the numerical value of SpO₂ to be made to correspond to each piece of advice information may be increased. By doing this, also in the case where the decrease in SpO₂ is relatively small, “take rest” or “keep quiet” is selected and displayed.

However, in the case where the rising pace is too high, there is also a fear that the physical strength is rapidly lost in a short time and the user may fall into a dangerous state. Therefore, the notification information may be generated from a viewpoint different from the above viewpoint. As one example, a method in which in the case where the rising pace is too high, advice information for encouraging the user to take a rest is generated, or alert information for notifying that the pace is too high is generated is considered.

Further, as the atmospheric pressure (oxygen partial pressure) decreases, the value of SpO₂ decreases even in the case of a healthy subject. Therefore, the range of SpO₂ to be made to correspond to the respective pieces of advice information may be changed according to the atmospheric pressure. In this case, as the atmospheric pressure decreases, the numerical value (for example, the upper limit value and the lower limit value of the range) of SpO₂ is decreased.

Further, when the weather is bad, the atmospheric pressure decreases, and therefore, also in this case, the value of SpO₂ decreases. Therefore, in this case, as the weather is worse, the numerical value of SpO₂ to be made to correspond to the respective pieces of advice information may be decreased. For example, in the case where information indicating any of the following states: “sunny”, “cloudy”, and “rainy” is acquired as the weather information, the range of SpO₂ to be made to correspond to a given piece of advice information becomes as follows: the value in the case of “sunny”>the value in the case of “cloudy”>the value in the case of “rainy”.

Further, it is possible to set an appropriate numerical range of the pulse rate, and in the case where the pulse rate is too high or too low, it is presumed that some kind of abnormality occurs in the user. For example, even in the case where the value of SpO₂ is the same, the load applied to the user varies depending on whether or not the pulse rate is in an appropriate range. When the pulse rate is in the appropriate range, a predetermined amount of oxygen can be supplied to the whole body even if the heart does not overwork. However, when the pulse rate is too high, it becomes difficult to supply sufficient oxygen unless the heart beats actively so much. That is, in the case where the pulse rate is too high, notification information attaching greater importance to safety may be generated as compared with the case where the pulse rate is in the appropriate range. For example, in the example of the above-mentioned four pieces of advice information, the advice information of “take rest” or “keep quiet” is made more likely to be selected. As one example, in the case where the pulse rate is not in the appropriate range, the numerical value of SpO₂ to be made to correspond to the respective pieces of advice information may be increased as compared with the case where the pulse rate is in the appropriate range. By doing this, in the case where the pulse rate is outside the appropriate range, even if the decrease in SpO₂ is relatively small, “take rest” or “keep quiet” is selected and displayed.

Although the example in which the pulse rate is used is shown here, other biological information may be used. In this case, for example, in the same manner as in the case of the pulse rate, the result of the determination as to whether or not the biological information to be used is in an appropriate range may be utilized. In the case where it is outside the appropriate range, notification information attaching greater importance to safety for the user is generated as compared with the case where it is in the appropriate range. That is, the range of SpO₂ may be set so that “take rest” or “keep quiet” is more likely to be selected as compared with the case where it is in the appropriate range.

Further, the personal data of a user such as age may be used for the processing. In general, as the age is higher, the physical strength is decreased. Therefore, as one example, in the case where the age is high, the range of SpO₂ may be set so that “take rest” or “keep quiet” is more likely to be selected as compared with the case where the age is low.

### 3.3 Utilization of History Information

Further, the history of various types of information may be used for the generation of notification information. For example, in the case of a user who climbs a mountain, by continuously using the wearable device 100 according to this embodiment, data when climbing a mountain a plurality of times are accumulated. Therefore, by generating notification information for mountain climbing thereafter based on the past history, it becomes possible to generate notification information more suitable for the subject user.

For example, the wearable device 100 includes the memory section 150 which stores history information of arterial oxygen saturation information, and the processing section 130 may generate notification information based on the history information.

As the information determined from the history information of the arterial oxygen saturation information, the standard value of SpO₂ described above with reference to FIGS. 10 and 11 is considered. As described above, not only the absolute value of SpO₂, but also the variation with respect to the standard value of SpO₂ which is the normal SpO₂ value of the subject user become important index values. In that regard, it becomes possible to determine the standard value by storing the history information of the arterial oxygen saturation information. Further, information obtained from the history information of the arterial oxygen saturation information is not limited to the standard value. Even other form of information becomes information reflecting the personal characteristics of the subject user as long as the history information of the arterial oxygen saturation information which is the accumulation of actually measured values is used. Therefore, by using the history information, the effect of individual differences among users is suppressed, and appropriate notification information can be generated.

Although the history information of the arterial oxygen saturation information alone is useful, by associating the history information with other information, a more variety of processing can be performed. For example, the history information is information in which each piece of arterial oxygen saturation information and environmental information are associated with each other. In this case, the memory section 150 stores information in which each piece of arterial oxygen saturation information is associated with environmental information as the history information, and the processing section 130 may generate notification information based on the arterial oxygen saturation information associated with the environmental information.

For example, the value of SpO₂ as the history information and the altitude when the value is acquired are made to correspond to each other and stored. By doing this, the user can store the characteristic of the subject user such that when the altitude was about X (m), the value of SpO₂ was about Y (%). Due to this, the physical condition of the user can be estimated by comparison between the value Y′ (%) of SpO₂ when reaching a spot at an altitude X (m) in the latest mound climbing and the actual value Y in the past of the subject user stored as the history information.

For example, when Y′≧Y, the SpO₂ equal to or higher than the actual value in the past can be maintained also in the current mountain climbing, and it is considered that there is no problem in the physical condition. On the other hand, when Y′<Y, in the current mountain climbing, the degree of decrease in SpO₂ is greater than the past mountain climbing, and therefore, it can be determined that the physical condition is tends to be poor. Further, it is also possible to estimate how poor the physical condition is according to the degree of dissociation between Y′ and Y. The processing section 130 generates notification information so as to encourage the user to slow down, take a rest, or keep quiet earlier if the physical condition tends to be poor.

The information to be made to correspond to the arterial oxygen saturation information may be environmental information other than the altitude, or may be information other than the environmental information. For example, the history information may be information in which each piece of arterial oxygen saturation information, environmental information, and the biological information of a user are associated with one another. In this case, the memory section 150 stores information in which each piece of arterial oxygen saturation information is associated with environmental information and the biological information of a user as the history information, and the processing section 130 may generate notification information based on the arterial oxygen saturation information associated with environmental information and the biological information of a user.

For example, the value of SpO₂ as the history information and the altitude and pulse rate when the value is acquired are made to correspond to one another and stored. By doing this, the user can store the characteristic of the subject user such that when the altitude was about X (m), the value of SpO₂ was about Y (o), and the pulse rate was about Z (beats/min). Therefore, the processing section 130 can determine whether or not the physical condition of the user tends to be poor not only by comparison between the above-mentioned Y′ and Y, but also using Z which is the history of the pulse rate. The processing section 130 may change the content of notification information or notification timing based on the determination result.

For example, an evaluation function E (X, Y, Z, Y′, Z′) using X, Y, and Z, which are the history information, and Y′, which is the current value of SpO₂, and Z′, which is the current pulse rate, as parameters (variables) is set beforehand, and the physical condition of the user may be estimated based on the magnitude of the value of the evaluation function. Other than these, various modifications can be made to the processing by the processing section 130 based on the history information.

Further, whether the actual physical condition is good or poor also partially depends on the subjectivity of the user. Therefore, in the case where symptoms of altitude sickness such as headache are felt during mountain climbing, the user may perform input to the wearable device 100. The memory section 150 of the wearable device 100 stores that the user felt ill health at the timing when the input was performed, and in the generation of notification information thereafter, the processing is performed using the stored information.

For example, it is assumed that the memory section 150 stores that an input was made by the user when the altitude was X, the value of SpO₂ was Y, and the pulse rate was Z. In this case, the set of values (X, Y, Z) is not standard values for the subject user, but values indicating a dangerous state such that the user feels ill health. Therefore, in the case where a similar tendency is observed at a timing thereafter, it is determined that the physical condition of the user tends to be poor, and notification information is generated so as to encourage the user to take a rest earlier.

As described above, a case where the user is aware of ill health corresponds to a case where the user has been in a hypoxic state for a rather long period of time. Also from the viewpoint of preventing serious symptoms, the wearable device 100 desirably notifies the user of alert information or advice information before approaching the danger values of (X, Y, Z) stored. For example, simply, based on the history information that the user felt ill health at an altitude of about X (m), the processing section 130 may perform processing such that alert information is generated and output at a timing before the altitude in the latest mountain climbing reaches X (m). Similarly, the processing section 130 may generate and output alert information before the SpO₂ decreases to around Y, or before the pulse rate changes to around Z.

FIG. 12 shows one example of the history information to be stored in the memory section 150. In the example in FIG. 12, the value of SpO₂, altitude, pulse rate, and information indicating the presence or absence of input from a user (0: absence of input, 1: presence of input) acquired at a given timing tᵢ (i=0, 1, . . . ) are stored. For example, in an example shown in the first line, the following values: SpO₂=95.5, altitude=100, pulse rate=85, user input=0 are acquired at a timing of t₀, and made to correspond to one another and stored.

Incidentally, the acquisition timing (acquisition rate or calculation rate) of each type of information such as SpO₂, altitude, and pulse rate is not necessarily the same. Therefore, the history information to be stored in the memory section 150 is not limited to information in which the arterial oxygen saturation information is always associated with each one of the environmental information and the biological information. For example, there may be a case where a given value of SpO₂ is associated with the pulse rate but is not associated with the altitude. Further, the data to be associated is not limited to data for which the acquisition timing is the same, and data for which the acquisition timing is close may be associated with each other. For example, in the case where there were SpO₂ acquired at a time of T1 and the altitude acquired at a time of T2, when the following relationship: |T1−T2|≦δ (δ is a given threshold) or the like is satisfied, these two pieces of data are associated with each other and may be stored as the history information.

Other than these, with respect to specific examples of the history information to be stored in the memory section 150, and specific examples of the notification information generation processing to be executed by the processing section 130 using the history information, various modifications can be made.

### 4. Example of Display Screen

Finally, specific examples of the display screen when the notification information is displayed on the display section will be described. Hereinbelow, an example in which the output section 140 of the wearable device 100 is the display section 50 will be described, however, the display section which displays the notification information may be provided in the portable terminal device 300, the information processing device 400, or the like.

As described above, the arterial oxygen saturation information serves as an index value which indicates the amount of oxygen capable of being taken in the body, therefore, it is useful to present the arterial oxygen saturation information to a user who performs an activity at a high altitude. Further, by displaying notification information such as advice information or alert information, information presentation which is easier to understand can be made as compared with the case where only the value of SpO₂ is displayed, and therefore, it is also useful to display the notification information. In addition, a change in the surrounding environment of a user is a factor which has an influence on the physical condition of the user, and therefore, it is also useful to display the environmental information.

That is, the display section 50 which is the output section 140 may display the notification information, the environmental information, and the arterial oxygen saturation information. The display here may be a form in which the three pieces of information are displayed on one screen. Alternatively, the display section 50 can display a display screen of the notification information, a display screen of the environmental information, and a display screen of the arterial oxygen saturation information, and may display the three pieces of information by appropriately changing the display screen.

FIG. 13 shows an example of the screen displayed on the display section 50. As shown in FIG. 13, the value of SpO₂ (A1) which is the arterial oxygen saturation information, the value of the atmospheric pressure (A2) which is the environmental information, the value of the pulse rate (A3) which is the biological information, and advice information (A4) associated with the arterial oxygen saturation information, which is the notification information, are displayed on the display screen.

In the example in FIG. 13, the following four advice contents: “optimum pace” “slow down”, “take rest”, and “keep quiet” are displayed side by side in the vertical direction (A41), and also a bar graph is displayed next thereto (A42), and which of the four advice contents the current advice information notifies is displayed in a visible form. In FIG. 13, the bar graph shown in A42 is configured such that the display can be changed at finer levels than four levels. That is, even if the level is the same “slow down”, it is possible to distinguish whether the level is “slow down” close to “optimum pace” or “slow down” close to “take rest”. As described above, in the case where each piece of advice information and the range of SpO₂ are made to correspond to each other, by changing the display of the bar graph according to the value of SpO₂, display as shown in FIG. 13 can be made. In FIG. 13, the bar graph points out the position of A43, and it is found that the current advice information is information indicating “slow down” relatively close to “take rest”.

FIG. 14 shows another example of the screen displayed on the display section 50. In the same manner as in FIG. 13, FIG. 14 shows an example in which the value of SpO₂ (B1), the value of the atmospheric pressure (B2), the value of the pulse rate (B3), and advice information (B4, specifically, B41 and B42) are displayed. However, FIG. 14 is a display example at a timing different from FIG. 13, and the SpO₂ and the atmospheric pressure decrease and the pulse rate increases accompanying the activity of the user. Further, by this change, it is determined that the user has a risk of occurrence of symptoms of altitude sickness, and therefore, the bar graph B42 changes to a display pointing out the position indicated by B43, and it is found that the current advice information is “keep quiet”.

However, FIGS. 13 and 14 each show an example of the display screen, and various modifications can be made to a specific display screen.

Hereinabove, embodiments to which the invention is applied and modifications thereof are described, however, the invention is not limited to the respective embodiments and the modifications thereof intact, and can be embodied by modifying constituent elements without departing from the gist of the invention when it is practiced. Also, various inventions can be formed by appropriately combining a plurality of constituent elements disclosed in the respective embodiments and modifications described above. For example, some constituent elements may be omitted from all the constituent elements described in each embodiment or modification. Further, constituent elements described in different embodiments or modifications may be appropriately combined. In addition, in the specification or drawings, terms described together with different terms having the same meaning or a broader meaning at least once can be replaced with the different terms in any part of the specification or drawings. In this manner, various modifications and applications may be made without departing from the gist of the invention.

The entire disclosure of Japanese Patent Application No. 2016-039685, filed Mar. 2, 2016 is expressly incorporated by reference herein.

What is claimed is:

1. A wearable device, comprising:
an information acquisition section which acquires environmental information including at least one of location information of a user, atmospheric pressure information, and weather information;
a sensor which acquires arterial oxygen saturation information;
a processing section which generates notification information associated with an arterial oxygen saturation based on the environmental information and the arterial oxygen saturation information; and
an output section which outputs the notification information.

2. The wearable device according to claim 1, wherein the output section is a display section which displays at least one of advice information associated with the arterial oxygen saturation and alert information associated with the arterial oxygen saturation as the notification information.

3. The wearable device according to claim 2, wherein the display section displays the notification information, the environmental information, and the arterial oxygen saturation information.

4. The wearable device according to claim 2, wherein the advice information is information associated with the continuation of the exercise or the load of the exercise of the user.

5. The wearable device according to claim 2, wherein
the processing section determines which range of a first range to an N-th (N is an integer of 2 or more) range the arterial oxygen saturation falls in based on the arterial oxygen saturation information, and
when the arterial oxygen saturation is determined to fall in an i-th (i is an integer that satisfies the following relationship: 1≦i≦N) range, the processing section generates i-th advice information.

6. The wearable device according to claim 5, wherein the processing section changes the arterial oxygen saturation corresponding to at least one range of the first range to the N-th range according to the environmental information.

7. The wearable device according to claim 1, wherein the output section is a communication section which transmits alert information associated with the arterial oxygen saturation as the notification information.

8. The wearable device according to claim 1, wherein
the device further comprises a memory section which stores history information of the arterial oxygen saturation information, and
the processing section generates the notification information based on the history information.

9. The wearable device according to claim 8, wherein the history information is information in which each piece of arterial oxygen saturation information and the environmental information are associated with each other.

10. The wearable device according to claim 8, wherein the history information is information in which each piece of arterial oxygen saturation information, the environmental information, and biological information of the user are associated with one another.

11. A system, comprising:
the wearable device according to claim 1; and
a portable terminal device or an information processing device which makes a notification based on the notification information received from the wearable device.

12. A method for generating notification information using a wearable device, comprising:
acquiring environmental information including at least one of location information of a user, atmospheric pressure information, and weather information;
generating notification information associated with an arterial oxygen saturation based on arterial oxygen saturation information acquired from the user and the environmental information; and
outputting the notification information.

13. The method for generating notification information using a wearable device according to claim 12, wherein in the outputting the notification information, at least one of advice information associated with the arterial oxygen saturation and alert information associated with the arterial oxygen saturation is displayed on a display section.

14. The method for generating notification information using a wearable device according to claim 13, wherein in the outputting the notification information, the notification information, the environmental information, and the arterial oxygen saturation information are displayed on the display section.

15. The method for generating notification information using a wearable device according to claim 13, wherein the advice information is information associated with the continuation of the exercise or the load of the exercise of the user.

16. The method for generating notification information using a wearable device according to claim 13, wherein
in the generating notification information, it is determined which range of a first range to an N-th (N is an integer of 2 or more) range the arterial oxygen saturation falls in based on the arterial oxygen saturation information, and
when the arterial oxygen saturation is determined to fall in an i-th (i is an integer that satisfies the following relationship: 1≦i≦N) range, i-th advice information is generated.

17. The method for generating notification information using a wearable device according to claim 16, wherein in the generating notification information, the arterial oxygen saturation corresponding to at least one range of the first range to the N-th range is changed according to the environmental information.

18. The method for generating notification information using a wearable device according to claim 12, wherein in the outputting the notification information, alert information associated with the arterial oxygen saturation is transmitted from a communication section as the notification information.

19. The method for generating notification information using a wearable device according to claim 12, wherein
the method further comprises storing history information of the arterial oxygen saturation information, and
in the generating notification information, the notification information is generated based on the history information.

20. The method for generating notification information using a wearable device according to claim 19, wherein the history information is information in which each piece of arterial oxygen saturation information and the environmental information are associated with each other.

21. The method for generating notification information using a wearable device according to claim 19, wherein the history information is information in which each piece of arterial oxygen saturation information, the environmental information, and biological information of the user are associated with one another.

22. A wearable device, wherein
environmental information including at least one of location information of a user, atmospheric pressure information, and weather information,
information associated with an arterial oxygen saturation of the user,
pulse wave information of the user, and
at least one of advice information associated with the arterial oxygen saturation and alert information associated with the arterial oxygen saturation
are displayed on a display section.

23. The wearable device according to claim 22, wherein the advice information is information associated with the continuation of the exercise or the load of the exercise of the user.

24. The wearable device according to claim 22, wherein the alert information is information associated with a pace of the user.
